(19) United States
(12) Patent Application Publication
(10) Pub. No.: US 2026/0345387 A1
(43) Pub. Date: Nov. 5, 2026

(54) VITRECTOMY HANDPIECES FOR OPHTHALMIC PROCEDURES

(71) Applicant: Alcon Inc., Fribourg (CH)

(72) Inventor: Nathaniel Reyes, Santa Ana, CA (US)

(21) Appl. No.: 19/632,638

(22) Filed: Mar. 30, 2026

Related U.S. Application Data
(60) Provisional application No. 63/798,084, filed on May 1, 2025.

(51) Int. Cl. A61F 9/007

(52) U.S. Cl. CPC A61F 9/00763

(57) ABSTRACT

In certain embodiments, a vitrectomy handpiece is provided that includes a housing and a cutting assembly. The cutting assembly extends distally from the housing, and includes an outer cutting member and an inner cutting member. The outer cutting member has a dimpled port that includes a first depression and a second depression laterally aligned with each other on opposite sides of a longitudinal axis of the vitrectomy handpiece. The inner cutting member is disposed within the outer cutting member and includes two ports axially aligned with each other and with the dimpled port, or a tab at a distal end of the inner cutting member. Actuation of the inner cutting member causes the inner cutting member to rotate relative to the outer cutting member and cut vitreous of an eye.

### INTRODUCTION

Microsurgical procedures frequently require precision cutting and/or removing of various body tissues. For example, certain ophthalmic surgical procedures require cutting and removing portions of the vitreous, a transparent jelly-like material that fills the posterior segment of the eye. When removing vitreous, ophthalmic surgeons typically utilize a vitrectomy handpiece to cut the vitreous into smaller fragments, and then suction the fragmented vitreous out of the eye. To cut the vitreous, an inner cutting member of the vitrectomy handpiece reciprocates and moves relative to a port of an outer cutting member.

### BRIEF SUMMARY

The present disclosure relates generally to ophthalmic surgical instruments, such as vitrectomy handpieces, for performing ophthalmic surgical procedures.

In certain embodiments, a vitrectomy handpiece is provided. The vitrectomy handpiece includes a housing, and a cutting assembly extending distally from the housing, the cutting assembly comprising an outer cutting member having a dimpled port, and an inner cutting member disposed within the outer cutting member, where actuation of the inner cutting member causes the inner cutting member to rotate relative to the outer cutting member and cut vitreous of an eye.

In certain embodiments, another vitrectomy handpiece is provided. The other vitrectomy handpiece includes a housing, and a cutting assembly extending distally from the housing, the cutting assembly comprising an outer cutting member having a dimpled port, and an inner cutting member disposed within the outer cutting member, the inner cutting member having two ports axially aligned with each other and with the dimpled port, where actuation of the inner cutting member causes the two ports of the inner cutting member to rotate relative to the dimpled port of the outer cutting member and cut vitreous of an eye.

In certain embodiments, yet another vitrectomy handpiece is provided. The other vitrectomy handpiece includes a housing, and a cutting assembly extending distally from the housing, the cutting assembly comprising an outer cutting member having a dimpled port, and an inner cutting member disposed within the outer cutting member, the inner cutting member having a tab at a distal end of the inner cutting member, where actuation of the inner cutting member causes the tab of the inner cutting member to rotate relative to the dimpled port of the outer cutting member and cut vitreous of an eye.

The following description and the related drawings set forth in detail certain illustrative features of one or more embodiments.

### BRIEF DESCRIPTION OF THE DRAWINGS

The appended figures depict certain aspects of the one or more embodiments and are therefore not to be considered limiting of the scope of this disclosure.

FIG. 1 shows an example ophthalmic surgical system, according to certain embodiments.

FIG. 2 shows a block diagram of the example ophthalmic surgical system shown in FIG. 1, according to certain embodiments.

FIG. 3 is a longitudinal cross-sectional view of an example vitrectomy handpiece, according to certain embodiments.

FIG. 4 is a transverse cross-sectional view of an example single vane actuator of the vitrectomy handpiece shown in FIG. 3, according to certain embodiments.

FIG. 5A is a transverse cross-sectional view of the single vane actuator of FIG. 4A with a vane in a first position, according to certain embodiments.

FIG. 5B is a transverse cross-sectional view of the single vane actuator of FIG. 4A with the vane in a second position, according to certain embodiments.

FIGS. 6A-6C show different views of an example outer cutting member of the vitrectomy handpiece of FIG. 3, according to certain embodiments.

FIG. 7A shows an isometric perspective view of an example inner cutting member, according to certain embodiments.

FIG. 7B shows a front perspective view of the inner cutting member of FIG. 7A disposed within the outer cutting member shown in FIGS. 6A-6C, according to certain embodiments.

FIG. 8A shows an isometric perspective view of another example inner cutting member, according to certain embodiments.

FIG. 8B shows a front perspective view of the inner cutting member of FIG. 8A disposed within the outer cutting member shown in FIGS. 6A-6C, according to certain embodiments.

FIG. 9A shows an isometric perspective view of another example inner cutting member, according to certain embodiments.

FIG. 9B shows a front perspective view of the inner cutting member of FIG. 9A disposed within another example outer cutting member, according to certain embodiments.

FIG. 9C shows a cross-sectional side view of the inner cutting member disposed within the outer cutting member shown in FIG. 9B, according to certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

It will be readily understood that the components of the embodiments as generally described herein and illustrated in the appended Figures can be arranged and designed in a wide variety of different configurations. Thus, the following more detailed description of various embodiments, as represented in the Figures, is not intended to limit the scope of the present disclosure but is merely representative of various embodiments. While the various aspects of the embodiments are presented in the Figures, the Figures are not necessarily drawn to scale unless specifically indicated.

Reference throughout this specification to the term “distal” refers to a system, device, component, end, portion, or segment that is disposed closer to a patient and/or further from a console during an ophthalmic procedure; and the term “proximal” refers to the system, device, component, end, portion, or segment that is disposed further from the patient and/or closer to the console during the ophthalmic procedure.

The vitreous body, often referred to as the vitreous humor or simply “the vitreous,” is a transparent, colorless, and gelatinous mass that fills the space between the lens and the retina of the eyeball (e.g., a posterior segment of the eye). The vitreous makes up about 80% of the volume of the eyeball and helps maintain the round shape of the eye. Additionally, the vitreous assists in absorbing external mechanical shocks to the eye, provides nutrients to the lens, and supports the retina. The vitreous is mostly comprised of water with trace amounts of collagen and hyaluronic acid, which provide the vitreous with its gelatinous structure.

Microsurgical cutting handpieces used in ophthalmic surgeries that involve removal of the vitreous may include a hollow outer cutting member, a hollow inner cutting member arranged coaxially with and movably disposed within the hollow outer cutting member, a port extending radially through the outer cutting member near the distal end of the outer cutting member, and a port extending radially through the inner cutting member near the distal end of the inner cutting member. Vitreous humor and/or membranes may be aspirated into the open port of the outer cutting member and the inner cutting member may be actuated to extend distally. As the inner cutting member extends distally, cutting surfaces on both the inner and outer cutting members may cooperate to cut the vitreous and/or membranes, and the cut tissue may then be aspirated away through the inner cutting member. Vitreous and/or membranes may then be aspirated into the open ports of both the outer and inner cutting members, and the inner cutting member may be actuated to retract proximally. The inner and outer cutting members may cooperate to again cut vitreous and/or membranes and aspirate the cut tissue away. Typically, so that the cutting members cooperate to cut the vitreous and/or membranes, the inner cutting member is bent along its longitudinal axis (e.g., towards a port of the outer cutting member) to contact the outer cutting member and cut the vitreous at the port.

The embodiments described herein provide vitrectomy handpieces having an inner cutting member that rotationally oscillates relative to an outer cutting member having a dimpled port (e.g., a port having two or more depressions formed in an outer surface of the outer cutting member). The vitrectomy handpieces described herein are robust and provide increased cut-rates at low operating temperatures, which thereby facilitates quicker, and more efficient removal of vitreous from an eye while also improving the longevity of such handpieces and assemblies.

The vitrectomy handpieces described herein reduce the likelihood of breakage during manufacturing and operation, which decreases costs associated with vitrectomy handpieces. For example, so that the cutting members cooperate to cut the vitreous and/or membranes, the inner cutting member contacts the dimpled port of the outer cutting member to cut the vitreous at the port. Because the inner cutting member contacts the dimpled port of the outer cutting member during a limited portion of the inner cutting member’s cycle (i.e., rotation and retraction of the inner cutting member), less unwanted heat is produced from friction during reciprocation of the inner cutting member. As a result, the integrity of the inner cutting member is maintained, risk of breakage during reciprocation is reduced, longevity of the vitrectomy handpiece is increased, and less energy may be required to reciprocate the inner cutting member within the outer cutting member.

Further, the vitrectomy handpieces described herein can cut twice per cycle (i.e., twice per rotation and retraction of the inner cutting member) and may require less cycles to sufficiently cut the vitreous. This results in shorter surgery times and reduced energy consumption, which increase overall efficiency of ophthalmic procedures and decrease patient-risk. As such, less time and energy may be needed to complete ophthalmic procedures.

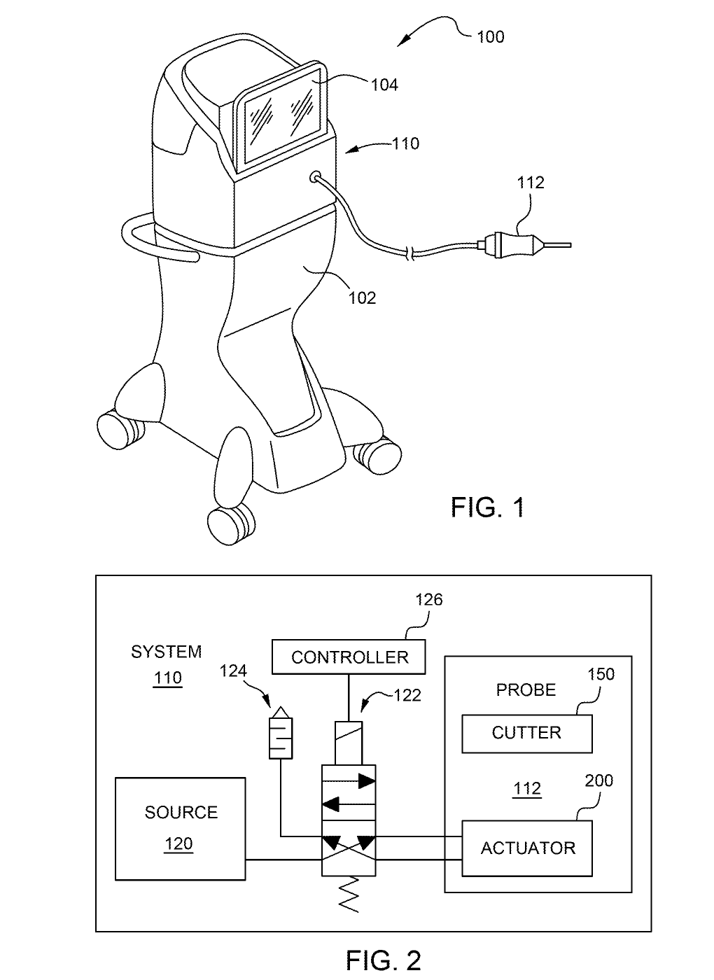

FIG. 1 shows an example ophthalmic surgical system 100, according to certain embodiments. The surgical system 100 includes a base housing 102 and an associated display screen 104 showing data relating to system operation and performance during a vitrectomy surgical procedure. The surgical system 100 includes a vitrectomy handpiece system 110 that includes a vitrectomy handpiece 112 (referred to herein as “handpiece 112”), as will be discussed in more detail below with respect to subsequent figures.

FIG. 2 shows example components of the handpiece system 110 of the example ophthalmic surgical system 100 shown in FIG. 1, according to certain embodiments. The handpiece system 110 includes the handpiece 112, a pneumatic pressure source 120, a handpiece driver (shown as an adjustable directional on-off pneumatic driver 122), a muffler 124, and a controller 126. The controller 126 may be a processor that includes one or more processing cores capable of performing parallel or sequential operations. Alternatively, the controller 126 may be a dedicated piece of hardware such as an application specific integrated circuit (ASIC). The pneumatic pressure source 120, the driver 122, the muffler 124, and the handpiece 112 are in fluid communication with each other along lines representing flow paths or flow lines. The controller 126 is in electrical communication with the driver 122. In some implementations, the controller 126 controls operation of both the driver 122 and various aspects of the handpiece 112, including the frequency of oscillation by way of a pneumatic vane actuator 200 as well as a flow rate of fluid to and from the surgical site.

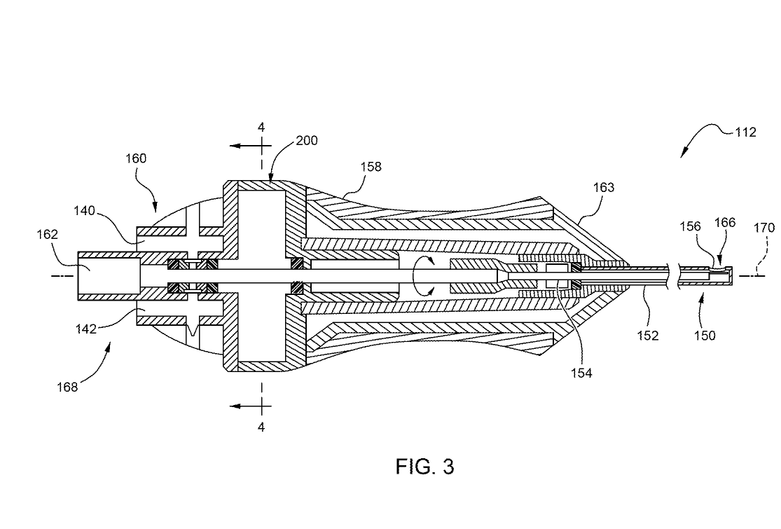

FIG. 3 is a longitudinal cross-sectional view of an example vitrectomy handpiece, such as the handpiece 112 of FIG. 1, according to certain embodiments. The handpiece 112 includes a housing 158 that is sized and shaped for grasping by a hand of a user and a cutting assembly 150 configured for insertion into the eye of a patient. The cutting assembly 150 extends from a distal portion 163 of the housing 158 along a longitudinal axis 170 of the handpiece 112. The cutting assembly 150 includes an outer cutting member 152 coupled to and extending from the housing 158. A dimpled port 156 that is sized and shaped to receive tissue within the eye is formed within the outer cutting member 152 at a distal end 166 of the handpiece 112. The cutting assembly 150 also includes an inner cutting member 154 shown in a non-sectional side view. The inner cutting member 154 may be rotatably disposed within the outer cutting member 152. In that regard, the inner cutting member 154 is configured to rotate about its longitudinal axis, which in this implementation, is coaxial with the longitudinal axis 170. The inner cutting member 154 is driven by the pneumatic vane actuator 200 positioned within the housing 158. In this example, the handpiece 112 is a pneumatically driven handpiece that operates by receiving pneumatic pressure alternating through a first port 140 and a second port 142 (ports 140, 142). To provide aspiration of materials from the cutting assembly 150, the housing 158 includes an end piece 160 with the ports 140, 142 and one suction port 162 at a proximal end 168 of the handpiece 112.

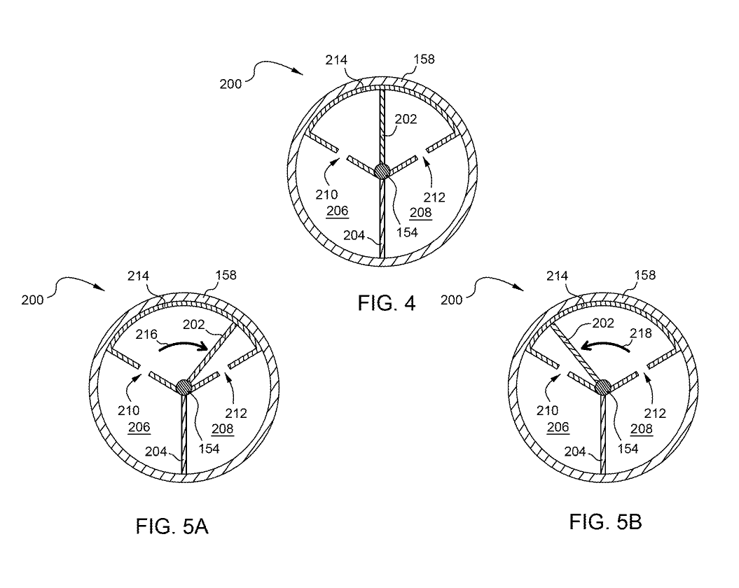

FIG. 4 is a transverse cross-sectional view of an example single vane actuator, such as the pneumatic vane actuator 200 of the handpiece 112 shown in FIG. 3, according to certain embodiments. FIG. 4 is a cross-sectional illustration of the pneumatic vane actuator 200 along section line 4-4 of FIG. 3. The pneumatic vane actuator 200 is a single vane actuator including a vane 202 that moves in response to air pressure and/or vacuum (negative) pressure within the chambers 206, 208. In that regard, the air pressure is applied across a surface of the vane 202, which urges the vane 202 to rotate. The vane 202 is mechanically coupled to the inner cutting member 154 such that rotation of the vane 202 causes corresponding rotation of the inner cutting member 154. The chambers 206, 208 are isolated from one another by a seal 204. The vane 202 is positioned within an actuator housing 214. The actuator housing 214 includes inlets 210, 212 that direct air pressure respectively from the chambers 206, 208 to the vane 202. In that regard, the chamber 206 is in fluid communication with the first port 140 (shown in FIG. 3) and the chamber 208 is in fluid communication with the second port 142 (also shown in FIG. 3). Thus, air pressure that is received at the first port 140 is transmitted to the vane 202 via the chamber 206 and the inlet 210. Air pressure that is received at the second port 142 is transmitted to the vane 202 via the chamber 208 and the inlet 212.

FIG. 5A is a transverse cross-sectional view of the pneumatic vane actuator 200 of FIG. 4 with the vane 202 in a first position, according to certain embodiments. FIG. 5B is a transverse cross-sectional view of the pneumatic vane actuator 200 of FIG. 4 with the vane 202 in a second position, according to certain embodiments. Accordingly, FIGS. 5A-5B are described together herein for clarity purposes.

FIGS. 5A-5B show the pneumatic vane actuator 200 in different operating positions. The operation of the pneumatic vane actuator 200 is described with reference to FIGS. 2 and 5A-5B. The driver 122 (shown in FIG. 2) may be a standard four-way on-off valve. The on-off pneumatic driver 122 may be configured to receive operating signals from the controller 126. The driver 122 may have a solenoid or moving magnet valve that operates to move the driver to one of the two on-off positions depicted in the example of FIG. 2.

Referring to FIGS. 2 and 3 the driver 122 is in a position to provide pneumatic pressure to the first port 140 and to vent pneumatic pressure from the second port 142. In this position, pneumatic pressure may pass from the pneumatic pressure source 120, through the on-off pneumatic driver 122, and to the first port 140 where the pneumatic pressure provides pneumatic power to the handpiece 112. In particular, the pneumatic pressure acts on the vane 202 via the chamber 206 and the inlet 210 to rotate the vane in a first direction 216, as illustrated in FIG. 5A. The inner cutting member 154 is correspondingly rotated in the first direction 216. For example, the first direction 216 may be a clockwise or counterclockwise direction. At the same time, pneumatic pressure at the second port 142 from the chamber 208 may pass through the on-off pneumatic driver 122 to the muffler 124 where it is exhausted, for example, to the atmosphere. In some instances, vacuum pressure is applied to the vane 202 via the chamber 208 and the second port 142 at the same time as pneumatic pressure is applied in the chamber 206 and the first port 140. The pneumatic pressure and vacuum pressure cooperate to rotate the vane 202 and the inner cutting member 154 in the first direction 216.

In the other position, the on-off pneumatic driver 122 allows pneumatic pressure to pass from the pneumatic pressure source 120 (as shown in FIG. 2) to the second port 142 (as shown in FIG. 3), where the pneumatic pressure provides pneumatic power to the handpiece 112. In particular, the pneumatic pressure acts on the vane 202 via the chamber 208 and the inlet 212 to rotate the vane in a second direction 218, as illustrated in FIG. 5B. The inner cutting member 154 is correspondingly rotated in the second direction 218. For example, the second direction 218 may be the opposite of the first direction 216. At the same time, pneumatic pressure at the first port 140 from the chamber 206 may vent through the on-off pneumatic driver 122 to the muffler 124 where it is exhausted to the atmosphere. In some instances, vacuum pressure is applied to the vane 202 via the chamber 206 and the first port 140 at the same time as pneumatic pressure is applied in the chamber 208 and the second port 142. The pneumatic pressure and vacuum pressure cooperate to rotate the vane 202 and the inner cutting member 154 in the second direction 218.

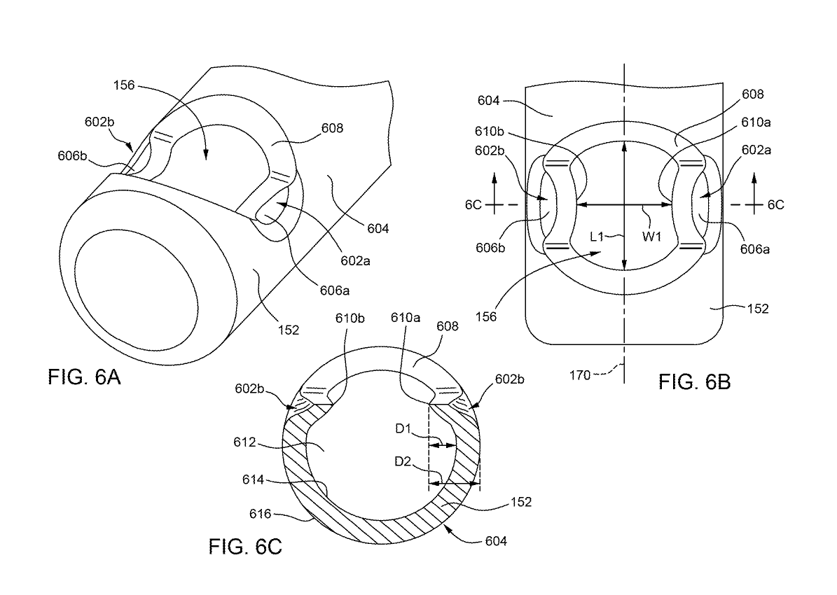

FIGS. 6A-6C show different views of an example outer cutting member, such as the outer cutting member 152 of the handpiece 112 of FIG. 3, according to certain embodiments. For example, FIG. 6A is an isometric perspective view of the outer cutting member 152, FIG. 6B is a front side view of the outer cutting member 152, and FIG. 6C is a transverse cross-sectional view of the outer cutting member 152. Accordingly, FIGS. 6A-6C are described together herein for clarity purposes.

The outer cutting member 152 includes the dimpled port 156 having a first depression 602a and a second depression 602b (depressions 602a-b) formed in an outer surface 604 of the outer cutting member 152. The first depression 602a and the second depression 602b include a first curved surface 606a and a second curved surface 606b (curved surfaces 606a-b), respectively. The depressions 602a-b are symmetrically disposed or formed relative to the longitudinal axis 170. In other words, the depressions 602a-b are both laterally aligned with each other on opposite sides of a longitudinal axis 170 of the handpiece 112, as shown in FIG. 6B. The depressions 602a-b also extend towards each other from an outer circumference 616 of the outer cutting member 152, as shown in FIG. 6C.

The depressions 602a-b may be formed by inserting a solid rod into a cavity 612 (shown in FIG. 6C) of the outer cutting member 152 and pressing against opposing sides of the outer surface 604 with a mechanical press. The solid rod has a diameter that is less than an inner diameter of the outer cutting member 152, which allows the outer surface 604 to deform and curve inwards when engaged by the mechanical press. By engaging the outer surface 604 while the solid rod is disposed within the outer cutting member 152, rounded ends of the mechanical press form the depressions 602a-b. In certain embodiments, the depressions 602a-b may be formed before or after a portion of the outer cutting member 152 has been removed (or cut out) to form the dimpled port 156.

As shown in FIGS. 6A-6B, the dimpled port 156 further includes a port surface 608, which has a deformed ovular shape. As shown in FIG. 6B, the dimpled port 156 has a first length (L1) extending longitudinally (i.e., parallel with the longitudinal axis 170) and a first width (W1) extending laterally (i.e., orthogonal to the longitudinal axis 170). The first length (L1) may range from 0.22 millimeters (mm) to 0.55 mm (e.g., from 0.23 mm to 0.54 mm, 0.24 mm to 0.53 mm, or 0.25 mm to 0.53 mm) and the first width (W1) may range from 0.15 mm to 0.67 mm (e.g., 0.16 mm to 0.66 mm, 0.17 mm to 0.65 mm, or 0.18 mm to 0.64 mm). In certain embodiments, the first length (L1) is greater than, less than, or equal to the first width (W1).

Inside the port surface 608 is a first cutting edge 610a and a second cutting edge 610b (cutting edges 610a-b). The first cutting edge 610a and the second cutting edge 610b may also be referred to herein as a “first outer cutting edge” and a “second outer cutting edge” (outer cutting edges), respectively. The cutting edges 610a-b have a curved or semi-circular shape, as shown in FIG. 6B, but, in certain other embodiments, may each include a partial straight portion that is parallel to the longitudinal axis 170. The cutting edges 610a-b extend into a cavity 612 of the outer cutting member 152, as shown in FIG. 6C. For example, the cutting edges 610a-b extend into the cavity 612 from an inner circumference 614 and the outer circumference 616 of the outer cutting member 152.

The first cutting edge 610a extends from the inner circumference 614 by a first distance (D1) and from the outer circumference 616 by a second distance (D2). The second distance (D2) is greater than the first distance (D1). The second cutting edge 610b may extend from the inner circumference 614 and the outer circumference 616 by the same distances as the first cutting edge 610a.

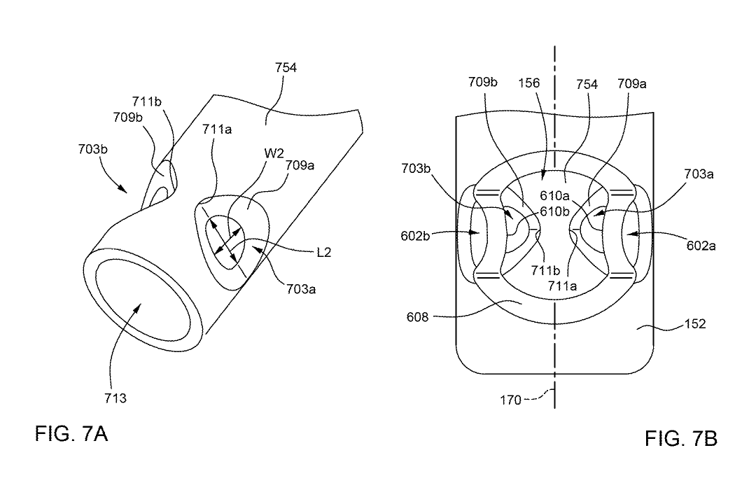

FIG. 7A shows an isometric perspective view of an example inner cutting member 754, according to certain embodiments. FIG. 7B shows a front perspective view of the inner cutting member 754 of FIG. 7A disposed within the outer cutting member 152 shown in FIGS. 6A-6C, according to certain embodiments. Accordingly, FIGS. 7A-7B are described together herein for clarity purposes.

The inner cutting member 754 can be implemented in the handpiece 112 and coupled with the outer cutting member 152 similar to the inner cutting member 154 described with respect to FIG. 3. Additionally, the inner cutting member 754 can be actuated via the pneumatic vane actuator 200 described with respect to FIGS. 4 and 5A-5B.

As shown in FIG. 7A, the inner cutting member 754 includes a first port 703a and a second port 703b (ports 703a-b) formed in the inner cutting member 754. In FIGS. 7A and 7B, the ports 703a-b each have an ovular shape but, in other embodiments, may instead have a circular shape, teardrop shape, or other similar shape. The ports 703a-b provide access to a cavity 713 that extends along an entire length of the inner cutting member 754. As such, tissue may be aspirated through at least one of the ports 703a-b and into the cavity 713.

The first port 703a has a second length (L2) extending laterally (i.e., orthogonal to the longitudinal axis 170) and a second width (W2) extending longitudinally (i.e., parallel with the longitudinal axis 170). The second port 703b generally has the same dimensions as the first port 703a, but, in certain embodiments, may instead have different dimensions. In certain embodiments, the second length (L2) may be greater than, less than, or equal to the second width (W2). In certain embodiments, the second length (L2) and/or the second width (W2) of the inner cutting member 754 are defined according to the first length (L1) and/or the first width (W1) of the dimpled port 156.

The ports 703a-b are axially aligned along a transverse axis of the inner cutting member 754 such that a straight line extends through both ports 703a-b. The first port 703a and the second port 703b include a first port surface 709a and a second port surface 709b (port surfaces 709a-b), respectively. The port surfaces 709a-b define oblique passageways for the ports 703a-b through the inner cutting member 754. The first port 703a and the second port 703b further include a first inner cutting edge 711a and a second inner cutting edge 711b (inner cutting edges 711a-b), respectively.

As shown in FIG. 7B, when the inner cutting member 754 is disposed within the outer cutting member 152, the ports 703a-b of the inner cutting member 754 are laterally aligned with the dimpled port 156 of the outer cutting member 152. And, the inner cutting edges 711a-b of the inner cutting member 754 are laterally aligned with the cutting edges 610a-b of the outer cutting member 152. As such, during actuation of the inner cutting member 754, the inner cutting edges 711a-b of the inner cutting member 754 engage with a corresponding one of the cutting edges 610a-b of the outer cutting member 152. For example, the first inner cutting edge 711a of the inner cutting member 754 engages with the first cutting edge 610a of the outer cutting member 152 and the second inner cutting edge 711b of the inner cutting member 754 engages with the second cutting edge 610b of the outer cutting member 152.

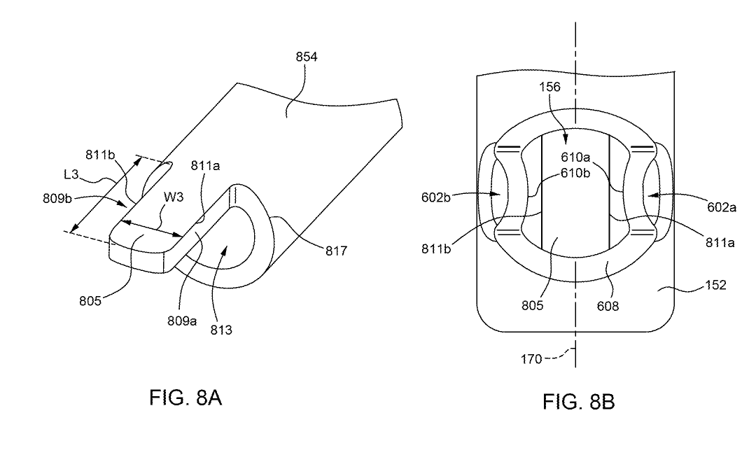

FIG. 8A shows an isometric perspective view of another example inner cutting member 854, according to certain embodiments. FIG. 8B shows a front perspective view of the inner cutting member 854 of FIG. 8A disposed within the outer cutting member 152 shown in FIGS. 6A-6C, according to certain embodiments. Accordingly, FIGS. 8A-8B are described together herein for clarity purposes.

The inner cutting member 854 can be implemented in the handpiece 112 and coupled with the outer cutting member 152 similar to the inner cutting member 154 described with respect to FIG. 3. Additionally, the inner cutting member 854 can be actuated via the pneumatic vane actuator 200 described with respect to FIGS. 4 and 5A-5B.

As shown in FIG. 8A, the inner cutting member 854 includes a distally extending tab 805 and a cavity 813 that extends proximally from a circumferential rim 817 along a length of the inner cutting member 854. The tab 805 has a substantially rectangular shape and extends in a direction that is parallel to the longitudinal axis 170. The tab 805 extends radially in a distal direction from a circumferential rim 817 of the inner cutting member 854. On opposing sides of the tab 805, the tab 805 includes a first surface 809a and a second surface 809b (surfaces 809a-b). A first inner cutting edge 811a and a second inner cutting edge 811b (inner cutting edges 811a-b) are formed along a perimeter of the first surface 809a and the second surface 809b, respectively. The inner cutting edges 811a-b of the inner cutting member 854 engage the cutting edges 610a-b of the outer cutting member 152 similar to the inner cutting edges 711a-b of the inner cutting member 754 described with respect to FIGS. 7A-7B.

As shown in FIG. 8A, the tab 805 has a third length (L3) extending longitudinally (i.e., parallel with the longitudinal axis 170) and a third width (W3) extending laterally (i.e., orthogonal to the longitudinal axis 170). In certain embodiments, the third length (L3) and the third width (W3) of the inner cutting member 854 are defined according to the first length (L1) and the first width (W1) of the dimpled port 156. For example, the third length (L3) of the tab 805 may be greater than or equal to the first length (L1) of the dimpled port 156, such that the tab 805 extends across the first length (L1) of the dimpled port 156.

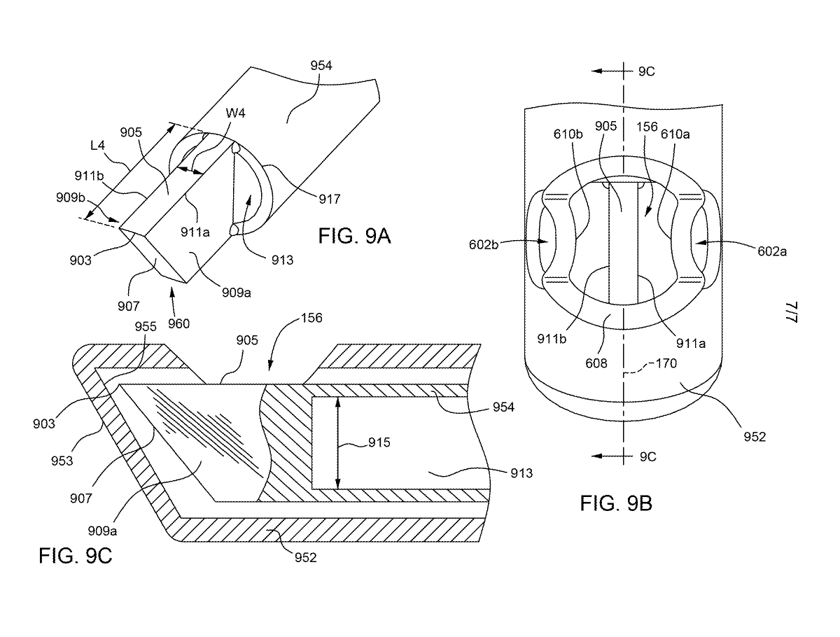

FIG. 9A shows an isometric perspective view of another example inner cutting member 954, according to certain embodiments. FIG. 9B shows a front perspective view of the inner cutting member 954 of FIG. 9A disposed within another example outer cutting member 952, according to certain embodiments. FIG. 9C shows a cross-sectional side view of the inner cutting member 954 disposed within the outer cutting member 952 shown in FIG. 9B, according to certain embodiments. Accordingly, FIGS. 9A-9C are described together herein for clarity purposes.

The inner cutting member 954 can be implemented in the handpiece 112 and coupled with the outer cutting member 952 similar to the inner cutting member 154 and the outer cutting member 152 described with respect to FIG. 3. Additionally, the inner cutting member 954 can be actuated similar to the inner cutting member 154, for example, via the pneumatic vane actuator 200 described with respect to FIGS. 4 and 5A-5B.

As shown in FIGS. 9A and 9C, the inner cutting member 954 includes a distally extending tab 905 and a cavity 913 that extends proximally from a circumferential rim 917 along a length of the inner cutting member 954. The tab 905 has a partially trapezoidal shape and extends in a direction that is parallel to the longitudinal axis 170. The tab 905 is formed along a diameter 915 of the inner cutting member 954. In other words, the tab 905 extends across the cavity 913 of the inner cutting member 154.

At a distal end 960 of the tab 905, the tab 905 includes a distal edge 903 and a beveled surface 907. The beveled surface 907 is angled at an angle that is non-normal relative the longitudinal axis 170. To accommodate the beveled surface 907, the outer cutting member 952 has a beveled tip 953 that is also angled non-normal relative to the longitudinal axis 170. As shown in FIG. 9C, when the inner cutting member 954 is positioned within the outer cutting member 952, there is a gap between the distal edge 903 of the inner cutting member 954 and an inner surface 955 of the outer cutting member 952. The gap between the distal edge 903 and the inner surface 955 allows for the inner cutting member 954 to rotate about the longitudinal axis 170 without contacting the inner surface 955 of the outer cutting member 952.

On opposing sides of the tab 905, the tab 905 includes a first surface 909a and a second surface 909b (surfaces 909a-b). A first inner cutting edge 911a and a second inner cutting edge 911b (inner cutting edges 911a-b) are formed along a perimeter of the first surface 909a and the second surface 909b, respectively. The inner cutting edges 911a-b of the inner cutting member 954 engage the cutting edges 610a-b of the outer cutting member 152 similar to the inner cutting edges 711a-b of the inner cutting member 754 described with respect to FIGS. 7A-7B.

The cutting edges 911a-b of the tab 905 have a fourth length (L4) extending longitudinally (i.e., parallel with the longitudinal axis 170) and the tab 905 has a fourth width (W4) extending laterally (i.e., orthogonal to the longitudinal axis 170). In certain embodiments, the fourth length (L4) and the fourth width (W4) are defined according to the first length (L1) and the first width (W1) of the dimpled port 156. For example, the fourth length (L4) may be greater than or equal to the first length (L1) of the dimpled port 156, such that the tab 805 extends across the first length (L1) of the dimpled port 156.

In certain embodiments, the beveled surface 907 of the inner cutting member 954 and the beveled tip 953 of the outer cutting member 952 may instead be perpendicular, or orthogonal to the longitudinal axis 170. In other words, the tab 905 of the inner cutting member 954 may have a flat distal surface and be implemented with the outer cutting member 152 shown in FIGS. 6A-6C.

In view of the embodiments described above with respect to FIGS. 6A-6C, 7A-7B, 8A-8B and 9A-6C, as a result of the depressions 602a-b being curved inwards, interference points are established when the inner cutting edges 711a-b, 811a-b, or 911a-b of the inner cutting member 754, 854, or 954 engage the cutting edges 610a-b of the outer cutting member 152 or 952. An “interference point” refers to an area where the inner cutting member 754, 854, or 954 and the outer cutting member 152 or 952 at least partially contact each other. During actuation of the inner cutting member 754, 854, or 954, the inner cutting member 754, 854, or 954 rotationally oscillates within the outer cutting member 152 or 952 as described with respect to FIGS. 3, 4, and 5A-5B. Thus, a first interference point is established at the first cutting edge 610a when the first inner cutting edge 711a, 811a, or 911a contacts the first cutting edge 610a and a second interference point is established at the second cutting edge 610b when the second inner cutting edge 711b, 811b, or 911b contacts the second cutting edge 610b.

When the inner cutting member 754, 854, or 954 rotationally oscillates within the outer cutting member 152 or 952, the inner cutting edges 711a-b, 811a-b, or 911a-b alternately engage with the cutting edges 610a-b. For example, the first inner cutting edge 711a, 811a, or 911a engages with the first cutting edge 610a when the inner cutting member 754, 854, or 954 rotates in the first direction 216 (shown in FIG. 5A) and then the second inner cutting edge 711b engages with the second cutting edge 610b when the inner cutting member 754, 854, or 954 rotates back in the second direction 218 (shown in FIG. 5B). In certain embodiments, when the inner cutting edges 711a-b, 811a-b, or 911a-b engage a corresponding one of the cutting edges 610a-b, the inner cutting edges 711a-b, 811a-b, or 911a-b contact and rotate at least partially past the cutting edges 610a-b, thereby forming a shearing force at the interference points (i.e., the cutting edges 610a-b).

The shearing force between the inner cutting edges 711a-b, 811a-b, or 911a-b and the cutting edges 610a-b allows the handpiece 112 to effectively cut tissue at the dimpled port 156 without undercutting the tissue. In other words, the shearing force between the inner cutting edges 711a-b, 811a-b, or 911a-b and the cutting edges 610a-b provides a clean cut by effectively separating the tissue at the interference points.

And, rotationally oscillating the inner cutting member 754, 854, or 954 within the outer cutting member 152 or 952 causes the inner cutting member 754, 854, or 954 to cut twice per cycle (i.e., twice per rotation and retraction of the inner cutting member). For example, the first inner cutting edge 711a, 811a, or 911a first cuts tissue at the first cutting edge 610a when the inner cutting member 754, 854, or 954 rotates in the first direction 216 (shown in FIG. 5A) and then the second inner cutting edge 711b cuts tissue at the second cutting edge 610b when the inner cutting member 754, 854, or 954 rotates back in the second direction 218 (shown in FIG. 5B).

Further, due to the arrangement of the ports 703a-b and the tabs 805, 905, vitreous can continuously be aspirated through the cavities 713, 813, and 913. That is, a pathway through the dimpled port 156 into the cavities 713, 813, and 913 is continuously provided during the rotational oscillation of the inner cutting members 754, 854, and 954. As an example, when the first inner cutting edge 711a of the inner cutting member 754 is in contact with the first cutting edge 610a of the outer cutting member 152, vitreous can be aspirated through the second port 703b, and vice-versa with the second inner cutting edge 711b and the first port 703a. As another example, when the first inner cutting edge 811a or 911a of the inner cutting member 854 or 954 is in contact with the first cutting edge 610a of the outer cutting member 152 or 952, vitreous can be aspirated past the second surface 809b, and vice-versa with the second inner cutting edge 811b or 911b and the first surface 909a.

Still further, because the dimpled port 156 of the outer cutting member 152 or 952 is deformed to have the depressions 602a-b, the cutting assembly 150 may be less likely to break in comparison to axial or guillotine style handpieces where the inner cutting member is deformed to create interference points. The dimpled port 156 may also reduce the amount of friction encountered during actuation of the handpiece 112 because the inner cutting member 754, 854, or 954 only contacts the outer cutting member 152 or 952 during cutting. In contrast, the inner cutting member of axial or guillotine style handpieces are in continuous contact with the outer cutting member.

The present disclosure may be embodied in other specific forms without departing from its spirit. The described embodiments are to be considered in all respects only as illustrative and not restrictive. The scope of the present disclosure is, therefore, indicated by the appended Claims rather than by this Detailed Description. All changes which come within the meaning and range of equivalency of the Claims are to be embraced within their scope.

Reference throughout this specification to features, advantages, or similar language does not imply that all the features and advantages that may be realized with the present disclosure should be or are in any single embodiment of the disclosure. Rather, language referring to the features and advantages is understood to mean that a specific feature, advantage, or characteristic described in connection with an embodiment is included in at least one embodiment of the present disclosure. Thus, discussions of the features and advantages, and similar language, throughout this specification may, but do not necessarily, refer to the same embodiment.

Furthermore, the described features, advantages, and characteristics of the disclosure may be combined in any suitable manner in one or more embodiments. One skilled in the relevant art will recognize, in light of the description herein, that the disclosure can be practiced without one or more of the specific features or advantages of a particular embodiment. In other instances, additional features and advantages may be recognized in certain embodiments that may not be present in all embodiments of the present disclosure.

Reference throughout this specification to “one embodiment”, “an embodiment”, or similar language means that a particular feature, structure, or characteristic described in connection with the indicated embodiment is included in at least one embodiment of the present disclosure. Thus, the phrases “in one embodiment”, “in an embodiment”, and similar language throughout this specification may, but do not necessarily, all refer to the same embodiment.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein but are to be accorded the full scope consistent with the language of the claims.

What is claimed is:

1. A vitrectomy handpiece, the vitrectomy handpiece comprising:
a housing; and
a cutting assembly extending distally from the housing, the cutting assembly comprising:
an outer cutting member having a dimpled port; and
an inner cutting member disposed within the outer cutting member,
wherein actuation of the inner cutting member causes the inner cutting member to rotate relative to the outer cutting member and cut vitreous of an eye.

2. The vitrectomy handpiece of claim 1, wherein the dimpled port comprises a first depression and a second depression laterally aligned with each other on opposite sides of a longitudinal axis of the vitrectomy handpiece.

3. The vitrectomy handpiece of claim 2, wherein the first depression and the second depression extend towards each other from an outer circumference of the outer cutting member.

4. The vitrectomy handpiece of claim 2, wherein:
the first depression comprises a first cutting edge, and
the second depression comprises a second cutting edge, and
wherein the first cutting edge and the second cutting edge extend into a cavity of the outer cutting member.

5. The vitrectomy handpiece of claim 1, wherein the dimpled port comprises an outer surface that curves inwards towards a cavity of the vitrectomy handpiece.

6. A vitrectomy handpiece, the vitrectomy handpiece comprising:
a housing; and
a cutting assembly extending distally from the housing, the cutting assembly comprising:
an outer cutting member having a dimpled port; and
an inner cutting member disposed within the outer cutting member, the inner cutting member having two ports axially aligned with each other and with the dimpled port,
wherein actuation of the inner cutting member causes the two ports of the inner cutting member to rotate relative to the dimpled port of the outer cutting member and cut vitreous of an eye.

7. The vitrectomy handpiece of claim 6, wherein:
each of the two ports of the inner cutting member comprises an inner cutting edge,
the dimpled port comprises two outer cutting edges, and
each of the inner cutting edges engages with a corresponding one of the two outer cutting edges during the actuation of the inner cutting member.

8. The vitrectomy handpiece of claim 6, wherein each of the two ports of the inner cutting member has an outer surface that define oblique passageways through the inner cutting member.

9. The vitrectomy handpiece of claim 6, wherein each of the two ports of the inner cutting member has a teardrop-shaped opening.

10. The vitrectomy handpiece of claim 6, wherein each of the two ports of the inner cutting member has an ovular-shaped opening.

11. A vitrectomy handpiece, the vitrectomy handpiece comprising:
a housing; and
a cutting assembly extending distally from the housing, the cutting assembly comprising:
an outer cutting member having a dimpled port; and
an inner cutting member disposed within the outer cutting member, the inner cutting member having a tab at a distal end of the inner cutting member,
wherein actuation of the inner cutting member causes the tab of the inner cutting member to rotate relative to the dimpled port of the outer cutting member and cut vitreous of an eye.

12. The vitrectomy handpiece of claim 11, wherein the tab extends radially in a distal direction from a circumferential rim of the inner cutting member.

13. The vitrectomy handpiece of claim 11, wherein the tab is formed along a diameter of the inner cutting member.

14. The vitrectomy handpiece of claim 11, wherein the tab extends across a length of the dimpled port.

15. The vitrectomy handpiece of claim 11, wherein:
the tab comprises two inner cutting edges,
the dimpled port comprises two outer cutting edges, and
each of the two inner cutting edges engage with a corresponding one of the two outer cutting edges during the actuation of the inner cutting member.
